(19) United States
(12) Patent Application Publication
(10) Pub. No.: US 2025/0213312 A1
(43) Pub. Date: Jul. 3, 2025

(54) A 4D-PRINTED HUMIDITY AND MAGNETIC DUAL RESPONSIVE ACTUATOR

(71) Applicant: City University of Hong Kong, Hong Kong (HK)

(72) Inventors: Jian LU, Hong Kong (HK); Yunhu HE, Hong Kong (HK); Zhengyi MAO, Hong Kong (HK); Yicheng HAN, Hong Kong (HK)

(21) Appl. No.: 18/399,594

(22) Filed: Dec. 28, 2023

(51) Int. Cl. A61B 34/30; B29C 64/118; B33Y 10/00; B33Y 70/00; B33Y 80/00

(52) U.S. Cl. CPC A61B 34/30; B29C 64/118; A61B 2034/301; A61B 2034/302; B33Y 10/00; B33Y 70/00; B33Y 80/00

(57) ABSTRACT

The present invention provides a 4D-printed humidity and magnetic dual responsive actuator with a bilayer structure, including a hydrogel film and a numerous of magnetic elastomer filaments, the numerous of magnetic elastomer filaments are printed on the hydrogel film to formed the bilayer structure, wherein the magnetic elastomer filaments are elastomer filaments embedded with magnetic particles, and the dual responsive actuator capable of responding to both humidity and magnetic fields, resulting in a reversible deformation that transforms into helix structures. The printed actuators can initially deform into helix structures in response to humidity stimuli and rapidly contract to a smaller size with the activation of a magnetic field. The shape deformation of this dual-responsive actuator is programmable and reversible, with stable and excellent repeatability.

### FIELD OF THE INVENTION

The present invention generally relates to the fields of smart responsive materials. More specifically, the present invention relates to dual responsive actuators (DRA) and direct 4D printing methods for fabricating the same.

### BACKGROUND OF THE INVENTION

In recent years, 4D printing has attracted enormous attention and has been extensively used in the study of various fields. Different categories of materials, including elastomers, hydrogels, metals, and ceramics, have been developed for 4D printing. A diverse range of applications has been achieved in areas such as actuators, soft robotics, and biomedical devices. Among these materials, hydrogels show excellent potential for biomedical applications due to their superior softness, wetness, responsiveness, biocompatibility, and bioactivity.

The 4D-printed hydrogel systems can be deformed to temporary shapes under the specific external stimuli. Humidity, light, heat, ions, pH and electricity are studied in the 4D printing of hydrogel materials to generate deformations. More recently, magnetic-driven 4D printing is being intensively investigated due to its significant advantages, such as fast response, untethered control and excellent biocompatibility. This magnetic-driven 4D printing offers a safe and effective manipulation method for biomedical applications, which typically require remote actuation in enclosed and confined spaces.

There are still two main challenges in the study of 4D-printed responsive hydrogel materials. First, the deformation speed is relatively slow due to the hydration and dehydration processes. Second, the combination design of 4D deformation and controlled untethered motivation in one integrated actuator is limited and difficult.

Therefore, there is a need for a technique to produce dual responsive actuators capable of being programmed for shape deformations and wireless locomotion. This invention addresses this need.

### SUMMARY OF THE INVENTION

The present invention provides a new strategy for 4D printing of dual-responsive actuators designed for remote navigation and untethered actuation. The structure can respond to both humidity and magnetic stimuli, deforming into various helix structures. The deformations into the helix structures are reversible and can be controlled by different parameters, including printing pathways and magnetic field intensity.

In a first aspect, the present invention provides a 4D-printed humidity and magnetic dual responsive actuator with a bilayer structure, including a hydrogel film and a numerous of magnetic elastomer filaments, the numerous of magnetic elastomer filaments are printed on the hydrogel film to formed the bilayer structure, wherein the magnetic elastomer filaments are elastomer filaments embedded with magnetic particles, and the dual responsive actuator capable of responding to both humidity and magnetic fields, resulting in a reversible deformation that transforms into helix structures.

In one of the embodiments, the hydrogel film inlcudes chitin, cellulose hydrogel, polyethylene glycol (PEG), poly (N-isopropyl acrylamide) (PNIPAM), polyvinyl alcohol (PVA), methacryloyl-modified gelatin (GelMA), or a combination thereof.

In one of the embodiments, the magnetic particles include neodymium-iron-boron (NdFeB) with a particle size of 5-100 μm.

In one of the embodiments, the elastomer filaments are polysiloxane materials comprising polydimethylsiloxane, polyborosiloxanes, or polycarbosiloxanes.

In one of the embodiments, when subjected to external magnetic field control, the dual responsive actuator demonstrates versatile functionality by seamlessly transitioning between two motion modes comprising rolling and wriggling.

In one of the embodiments, the swelling ratio between the hydrogel film and the magnetic elastomer filaments is 1.07:0.01.

In one of the embodiments, the dual-responsive actuator is designed for remote operation in a challenging environment comprising enclosed spaces, over high and sloped obstacles, and on viscous stomach surfaces.

In a second aspect, the present invention provides a method for fabrication a 4D-printed humidity and magnetic dual responsive actuator, including the steps of: preparing a numerous of magnetic elastomer filaments; printing the numerous of magnetic elastomer filaments on a hydrogel film with predesigned pattern and angles to form printed magnetic elastomer filaments; curing the printed magnetic elastomer filaments to form a bilayer structure; and magnetizing the bilayer structure with a predesigned helix structure to obtain the 4D-printed humidity and magnetic dual responsive actuator.

In one of the embodiments, the hydrogel film includes chitin, cellulose hydrogel, polyethylene glycol (PEG), poly (N-isopropyl acrylamide) (PNIPAM), polyvinyl alcohol (PVA), methacryloyl-modified gelatin (GelMA), or a combination thereof.

In one of the embodiments, the step of preparing a numerous of magnetic elastomer filaments including embedding magnetic particles into elastomer filaments.

In one of the embodiments, the elastomer filaments are polysiloxane materials comprising polydimethylsiloxane, polyborosiloxanes, or polycarbosiloxanes.

In one of the embodiments, the magnetic particles include neodymium-iron-boron (NdFeB) particles with a particle size of 5 μm to 100 μm.

In one of the embodiments, the weight ratio of the magnetic particles in the elastomer matrix ranges from 1 wt % to 20 wt %.

In one of the embodiments, the magnetic elastomer filaments have a printing angle on the hydrogel film ranging from 0° to 90°.

In one of the embodiments, the printed magnetic elastomer filaments have a diameter of approximately 100 to 500 μm.

In one of the embodiments, the curing temperature for the bilayer structure ranges from 60° C. to 150° C.

In one of the embodiments, the swelling ratio between the hydrogel film and the magnetic elastomer filaments is 1.07:0.01.

Due to the flexibility of the magnetic control, the dual-responsive actuator can be remotely operated in challenging environments, including enclosed spaces, over high and sloped obstacles, and on viscous stomach surfaces. The reconfiguration and locomotion functions endow these actuators with significant potential in applications related to soft robotics, smart actuators, and the biomedical field.

Compared to existing technologies, the present invention offers the following major advantages:
(1) The 4D-printed dual-responsive actuator can respond to both humidity and magnetic stimuli;
(2) Multiple helix structures can be achieved within a single actuator;
(3) The actuator's deformation can be programmed and designed through the arrangement of magnetic elastomer filaments on the hydrogel film;
(4) Under external magnetic field control, the actuator can achieve two distinct motion modes: rolling and wriggling;
(5) The actuator's rapid response and wireless control hold significant potential across diverse application fields and challenging environments; and
(6) The dual-responsive actuator enables drug loading and releasing capabilities.

### BRIEF DESCRIPTION OF THE DRAWINGS

Embodiments of the invention are described in more details hereinafter with reference to the drawings, in which:

FIG. 1A shows the fabrication process of the dual responsive actuator. FIG. 1B shows the swelling ratio of chitin hydrogel and magnetic elastomer. FIG. 1C shows the magnetic responsive of the cantilever sample in magnetic field. FIG. 1D shows the dual responsiveness of the 4D printed actuator. FIG. 1E shows the Young's modulus of chitin hydrogel and magnetic elastomer. FIG. 1F shows the Lap-shear adhesion curves of chitin/magnetic elastomer interface. FIG. 1G shows the Scanning Electron Microscope (SEM) image of chitin/magnetic elastomer interface;

FIG. 2 shows the hydration induced deformation of the actuator, and the effect of the printing angles of the magnetic elastomer filaments on the hydration induced deformation;

FIG. 3A shows pitches and curvatures of the actuator with different printing angles.

FIG. 3B shows the repeatability of hydration induced deformation;

FIG. 4 shows the magnetic stimuli induced deformation of the actuator, and the effect of the applied filed strength on the magnetic stimuli induced deformation;

FIG. 5A shows pitches and curvatures of the actuator with different filed intensities.

FIG. 5B shows the repeatability of the magnetic stimuli induced deformation;

FIG. 6A shows the rolling locomotion of the actuator under an applied magnetic field. FIG. 6B shows the wriggling locomotion of the actuator under an applied magnetic field;

FIG. 7 shows the remote navigation of the dual responsive actuator through a narrowed tunnel into an enclosed area underwater with the control of magnetic field;

FIG. 8 shows the locomotion capacity of the actuator on a high sloppy obstacle environment with complex structures;

FIG. 9 depicts the locomotion of the printed actuator on the real stomach surface which is immersed with simulated stomach fluids (with high viscosity);

FIG. 10A depicts the expansion process and the drug eluting function. FIG. 10B shows the expansion deformation of the dual responsive actuator under the change of magnetic field. FIG. 10C shows the drug releasing process with a time period of 30 minutes;

FIG. 11A shows the repeatability of the expansion deformation. FIG. 11B shows the area ratio of the vascular model before and after expansion; and

FIG. 12 shows the remote navigation of the dual responsive actuator in tortuous vascular structure under the control of external magnetic field.

### DETAILED DESCRIPTION

In the following description, 4D-printed dual responsive (humidity and magnetic field) actuators are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The present invention introduces a bilayer-designed 4D-printed dual-responsive actuator. Specifically, this actuator includes a hydrogel film and a numerous of magnetic elastomer filaments. These elastomeric filaments, which contain magnetic particles, are printed on the hydrogel film. This structural design enables the actuator to respond to both humidity and a magnetic field.

The dual-responsive deformation is achieved by hydrating the hydrogel film and magnetically actuating the elastomer filaments, respectively. The deformation under humidity stimuli is a result of the different expansion coefficients between elastomers and hydrogel films. The actuation under a magnetic field arises from the magnetization process of the actuators. The printed actuators can initially deform into helix structures in response to humidity stimuli and rapidly contract to a smaller size with the activation of a magnetic field. The degree of deformation can be regulated by the design of the printing pattern and the intensity of the external magnetic field. The shape deformation of this dual-responsive actuator is programmable and reversible, with stable and excellent repeatability.

In general, the hydrogel film can be expanded in humidity environment due to water absorption, a variety of hydrogel materials may be used including, but not limited to, chitin, cellulose hydrogel, polyethylene glycol (PEG), poly (N-isopropyl acrylamide) (PNIPAM), polyvinyl alcohol (PVA), or methacryloyl-modified gelatin (GelMA).

Preferably, the hydrogel film is a cellulose hydrogel film.

In one of the embodiments, the magnetic particles used in the fabrication of magnetic elastomers is neodymium-iron-boron (NdFeB), the size of NdFeB particles is around 5-100 μm.

In one of the embodiments, the swelling ratio between the hydrogel film and the magnetic elastomer filaments is 1.07:0.01.

In one of the embodiments, the size of NdFeB particles is around 5-20 μm.

Preferably, the size of NdFEB particles is approximately 10 μm. The weight ratio of NdFeB particles in the magnetic elastomer filaments ranges from 1 wt % to 20 wt %. For instance, the weight ratio of NdFeB particles in the magnetic elastomer filaments can be 1 wt %, 5 wt %, 10 wt %, 15 wt %, or 20 wt %.

In one of the embodiments, the elastomeric filaments can be selected from various kinds of polysiloxane materials, which may be polydimethylsiloxane, polyborosiloxanes and polycarbosiloxanes.

Depending on the selection of the elastomer material and the particle size of the magnetic particles, the higher printing pressure may need to be used for printing and the higher curing temperature may need to be used to complete curing of the polymer network.

In one embodiment, the dual responsive actuator was composed of the chitin film and magnetic/PDMS filaments. The weight ratio of NdFeB particles in the PDMS elastomer matrix ranges from 1 wt % to 5 wt %, and other fractions may be selected depending on the desired magnetic properties.

In one of the embodiments, after hydration, the filaments-film structure twists into a 3D tubular helix structure initially, and then, under the actuation of a magnetic field, the tubular helix structure can quickly contract to a smaller size.

The deformation of the helix structures is controllable by altering the printing angles of magnetic filaments on the hydrogel film. Additionally, the degree of contraction can be regulated by adjusting the intensity of the external magnetic field.

In one of the embodiments, the printing angles of magnetic filaments on the hydrogel film is in a range of 0° to 90°. For example, the printing angles of magnetic filaments on the hydrogel film can be 0°, 10°, 20°, 30°, 40°, 50°, 60°, 70°, 80°, or 90°.

In one of the embodiments, the dual responsive actuator can be precisely navigated through enclosed and confined spaces and shows great applicability to various harsh environments.

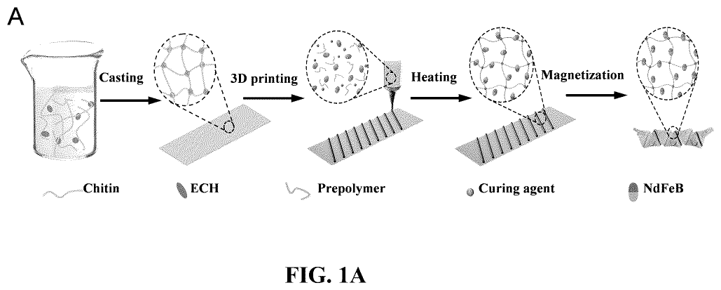

The present invention also provides a method for 4D printing of dual responsive actuators for remote navigation and untethered actuation. Four main processes are used in the 4D printed dual responsive actuator: (i) casting of the chitin film, (ii) printing of the magnetic elastomer filaments, (iii) heat treatment and curing of the magnetic elastomers, and (iv) magnetization of the actuator (FIG. 1A).

In one of the embodiments, the printed magnetic elastomer filaments have a diameter of approximately 100 to 500 μm. For example, 100 μm, 200 μm, 300 μm, 400 μm, or 500 μm.

In one of the embodiments, the curing temperature for the bilayer structure ranges from 60° C. to 150° C.

As shown in FIG. 1A, a bilayer structure was designed and printed. Firstly, a numerous of magnetic particles embedded elastomer filaments are prepared, and a chitin film is casted. Subsequently, the numerous of magnetic elastomer filaments are 3D-printed onto a hydrogel film (e.g., chitin film). Secondly, the printed flat sample is heated, cured and rolled to the predesigned smaller helix structure for magnetization. While two distinct helix structures have been depicted, alternative printing patterns or magnetization models can be employed based on the desired deformation characteristics of the dual responsive actuator.

After being immersed in water, the hydrogel film may start to swell due to hydration while the magnetic filaments remained the original size, resulting in the mismatch between the two layers. Thus, the 2D film twisted to the tubular structure.

In one embodiment, the method further including adding a curing agent. The weight ratio between the prepolymer and curing agent is 1:10-10:1.

In one embodiment, the thickness of the hydrogel film is in a range of 30-70 μm and the diameter of printed magnetic elastomer filaments is in a range of 100-300 μm.

Preferably, the thickness of the hydrogel film is 50 μm, and the diameter of printed magnetic elastomer filaments is 200 μm.

In one embodiment, the printing speed can be selected within a range of 5 mm/s to 20 mm/s. For instance, the printing speed can be 5 mm/s, 6 mm/s, 7 mm/s, 8 mm/s, 9 mm/s, 10 mm/s, 11 mm/s, 12 mm/s, 13 mm/s, 14 mm/s, 15 mm/s, 16 mm/s, 17 mm/s, 18 mm/s, 19 mm/s, or 20 mm/s.

In one embodiment, the printing pressure can be adjusted within a range of 2 MPa to 6 MPa. For instance, the printing pressure can be 2 MPa, 3 MPa, 4 MPa, 5 MPa, or 6 MPa.

In one embodiment, the curing temperature can be selected within a range of 60° C. to 150° C. For instance, the curing temperature can be 60° C., 70° C., 80° C., 90° C., 100° C., 110° C., 120° C., 130° C., 140° C., or 150° C.

### EXAMPLE

### Example 1—Material

NbFeB particles were purchased from Magnequench. PDMS SE 1700 was purchased from Dow Corning Corp. Commercial chitin powder was purchased from Golden-Shell Biochemical Co., Ltd. All other chemical reagents were purchased from J&K Scientific.

### Example 2—Preparation of a Hydrogel Film

The chitin powder was first purified to prepare the hydrogel film according to a previously reported method¹. Briefly, the commercial chitin powder was treated with 5 wt % NaOH solution for 10 hours, followed by 5 wt % HCl solution for 10 hours, then 5 wt % NaOH solution for another 10 hours under stirring. The chitin powder was washed with water and dried in an oven to produce the purified chitin powder after bleaching in a 4 wt % H₂O₂ solution for 13 hours. To prepare the chitin film, 4 g purified chitin powder was dispersed into 96 g NaOH/urea (11 wt %/4 wt %) aqueous solution to obtain a suspension. The suspension was then frozen at −30° C. for 3 hours before being thawed at room temperature with vigorous stirring. The freezing/thawing cycle was performed twice to get a clear chitin solution. Then, 3 g ECH was added into the chitin solution and placed at room temperature for 4 h for crosslink. The obtained chitin gel was washed by DI water to remove the unreacted chemicals. Finally, the chitin film was obtained by dry the chitin gel in air. The thickness of the hydrogel film is in a range of 60-1000 μm.

### Example 3—3D Printing of the Dual Responsive Actuators (DRA)

The hydrogel film was made according to EXAMPLE 2. The polydimethylsiloxane (PDMS) was used as the elastomer matrix in the below examples. The magnetic filaments were prepared by mixing 0.2 g NbFeB powder with 9.8 g PDMS (prepolymer: curing agent at weight ratio of 10:1) by planetary mixer at 2000 rpm for 2 minutes.

Next, the magnetic filaments were printed on the chitin film by a 3D printer (Regenovo Biotechnology Co. Ltd.). The printed and cured sample was used for the magnetization process. The sample was fixed under certain shape and magnetized by ASC Impulse Magnetizer. The printing speed is 5-30 mm/s. The printing pressure is 0.5-5 MPa. The curing temperature is 70-150° C.

### Example 4—Mechanical Tests and Characterization of a Dual Responsive Actuator

All tension and compression tests were conducted by Cellscale uniaxial tester. 100 N load cells was used to the tension tests with the stretch rate of 10 mm min⁻¹. 10 N load cell was employed for the compression test with loading/unloading rate of 10 mm min⁻¹. The interface between chitin film and magnetic filament was studied by SEM (Quanta 450 FEG, FEI). An Au layer was coated on the interface to increase the conductivity (Q150 TS) before SEM observation. All photos and videos were taken by a digital camera (Eos 5D Mark IV). The curvature of samples was determined by analysis software (Image J).

FIGS. 1B-1G showed the characterizations of the dual responsive actuator. After being immersed in water environment, the hydrogel film may start to swell due to hydration while the magnetic filaments remained the original size, resulting in the mismatch between the two layers. Thus, the 2D film twisted to the tubular structure. Turning to FIG. 1B, the swelling ratio data of chitin film and PDMS were measure. Swelling ratio is usually expressed as the ratio of the final volume to the initial volume. A swelling ratio of 0 indicates no change, while a swelling ratio greater than 0 indicates an increase in volume. The swelling ratio of chitin film was approximately 1.07 and the swelling ratio of PDMS was approximately 0.98, indicating that hydration behaviours of chitin hydrogel and PDMS elastomers were completely different.

Turning to FIG. 1C, the magnetic properties of the magnetic elastomer were measure. The unit “mT” stands for millitesla, which is a unit of magnetic flux density. The negative sign indicates the direction of the magnetic field. A cantilever sample was prepared with 2 wt % of NdFeB particles. The bending deformation of the sample under different intensities of actuating field was illustrated. The bending angle increased apparently with the higher magnetic field intensity.

The dual responsive actuation of the printed actuator was shown in FIG. 1D. After being immersed in water, the chitin film started to swell due to hydration while the magnetic filaments remained the original size, resulting in the mismatch between the two layers. Finally, the 2D film twisted to the tubular structure. Note that the shape transition could be fully recovered by placing the material back into ethanol for dehydration. Upon magnetic field actuation, the deformed tubular structure could rapidly transform into a pre-designed contracted helix shape and quickly reverted to its original form after the magnetic field was removed.

The mechanical properties of the chitin and magnetic elastomer were tested as shown in FIG. 1E. The modulus of chitin hydrogel exhibited over 19 times higher than that of PDMS elastomer, demonstrating the strong driving force for the helix structure deformation.

Turning to FIG. 1G, the SEM image showed that no gaps were observed at the interface, further revealing the tight adhesion between these two materials.

The adhesive strength between the PDMS elastomer filaments and the chitin film was measured and shown in FIG. 1F. The strength data extends to 8.3 N cm⁻², a critical factor for ensuring robust deformation.

### Example 5—Controlling of the Deformation in Dual Responsive Actuators

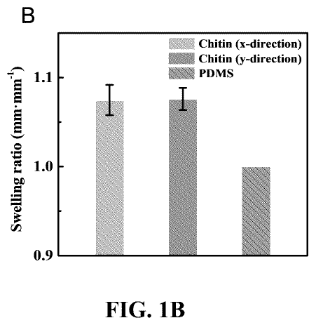

The effect of the printing angle and the magnetic field strength were systematically investigated. FIG. 2 showed the deformation of the dual responsive actuator under humidity stimuli, including the schematic of the hydration induced deformation. By adjusting the printing angles, the deformation turned from bending to twisting.

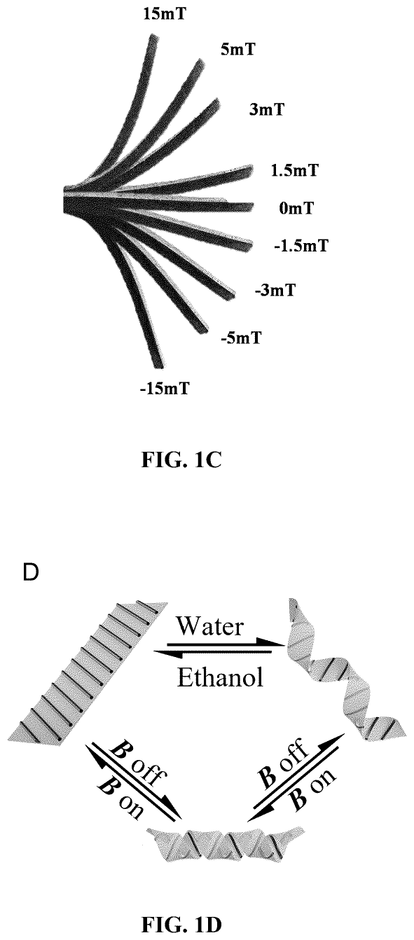

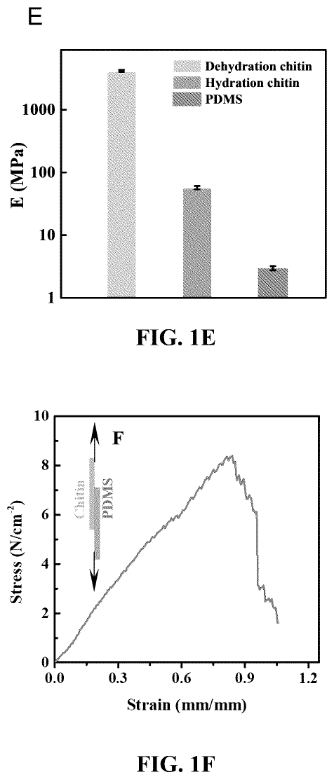

FIGS. 3A-3B showed the characterizations of the deformation behaviours under humidity stimuli. With increasing the printing angle from 0° to 45°, the pitch distance increased accordingly (FIG. 3A). Besides, the hydration induced deformation showed excellent stability and reversibility even after over 60 days of storage and 12 cycles (FIG. 3B).

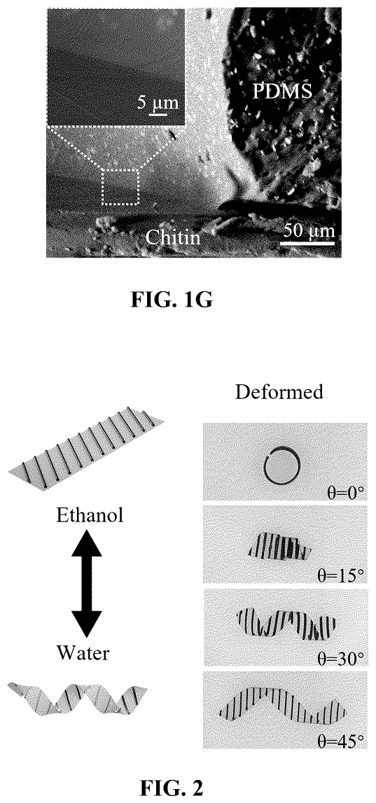

Moreover, the magnetic induced deformation was also studied. FIG. 4 showed the characterizations of the deformation behaviours under magnetic stimuli. Upon application of a uniform magnetic field, the dual responsive actuator deformed to a smaller size and fast recovered to its original shape after the removal of the magnetic field. The actuated actuator under field intensity of 3 mT, 6 mT, 9 mT, and 12 mT were shown.

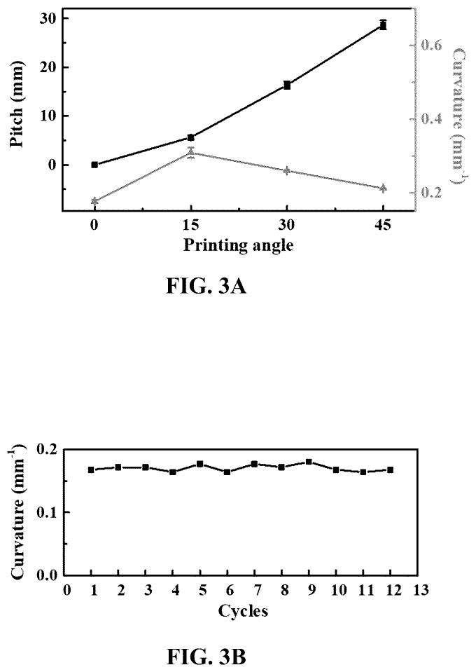

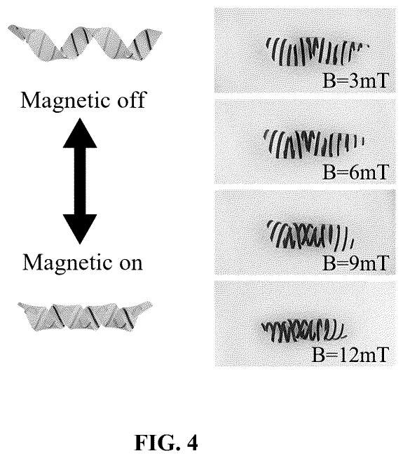

FIGS. 5A-5B showed the characterizations of the deformation behaviours under magnetic stimuli. The pitch distance and diameter of the dual responsive actuator decreased as increasing the field strength, demonstrating the feasibility of controlling the magnetic induced deformation (FIG. 5A). This rapid, reversible transformation could be repeated many times without decline due to the soft nature of the hydrogel and elastomer (FIG. 5B).

### Example 6—Remote Motivation of Dual Responsive Actuator in Harsh Environments

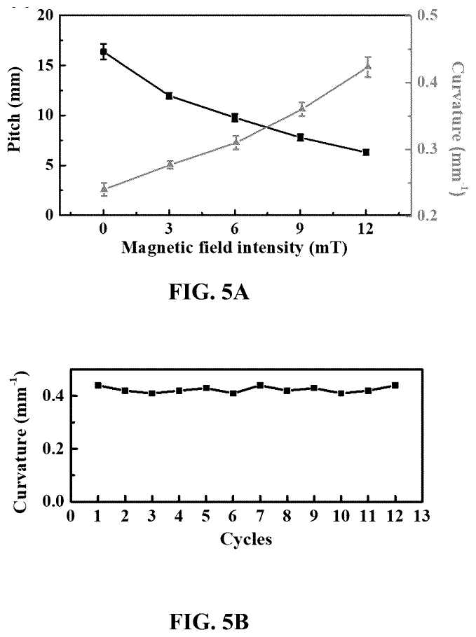

Due to the flexibility of magnetic control, remote operation of the dual-responsive actuator in harsh environments, such as enclosed spaces, high and sloped obstacles, and viscous stomach surfaces, can be easily achieved. FIGS. 6A-6B showed the two different locomotion modes of the dual responsive actuator under the external magnetic field. A NdFeB permanent magnet was used to create spatially varying fields for navigation and actuation. By rotating the magnetic field, the rolling movement of the actuator could be achieved. Another wriggling motion could be obtained by moving the actuating in the vertical and horizontal direction at the same time. The combination of different motivation models could improve the motion performance of the actuator in harsh environments.

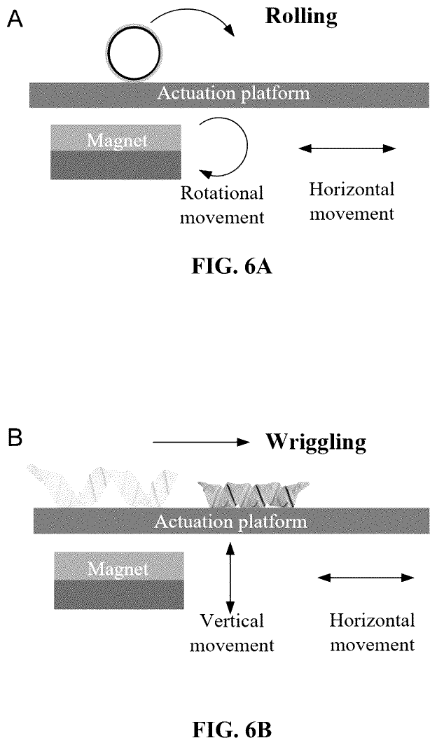

As shown in FIG. 7, the dual responsive actuator could be remotely navigated from an open space through the confined tunnel to the enclosed area with an average speed of 0.5 mm/s. Five steps were involved in this process: (i) deformation and rolling, (ii) turning, (iii) wriggling, (iv) crossing narrow tubes, and (v) rolling and releasing. The dual-responsive actuator underwent a sequence of steps: it was first deformed and rolled to the boundary of the open space, subsequently aligning itself with the tunnel direction. Following this, the actuator shrank to a smaller size by increasing the field strength, allowing it to wriggle through the narrowed tunnel. Finally, the actuator rolled to the designated position within the enclosed area.

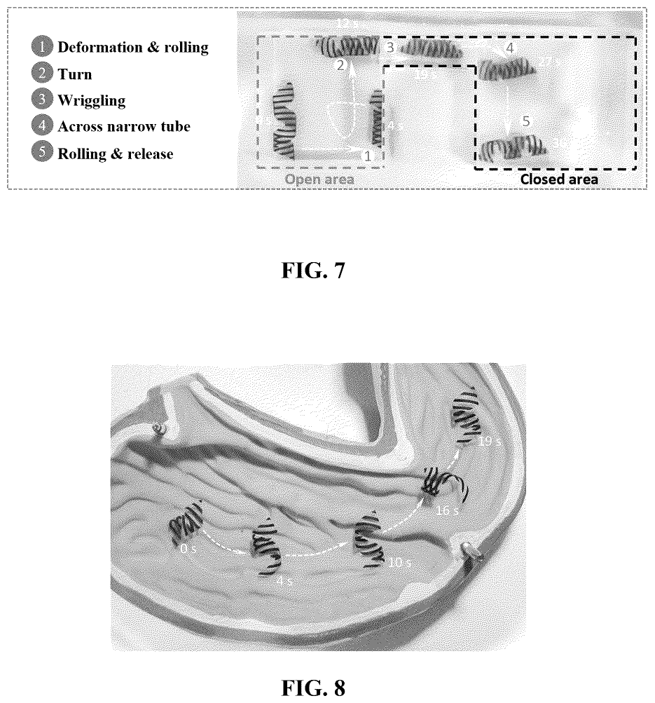

The locomotion ability of the dual responsive actuator was further demonstrated at high sloppy environment. FIG. 8 showed the movement in a stomach model with complex surface wrinkles. The actuator could reach to the target position with a predesigned pathway in 19 seconds.

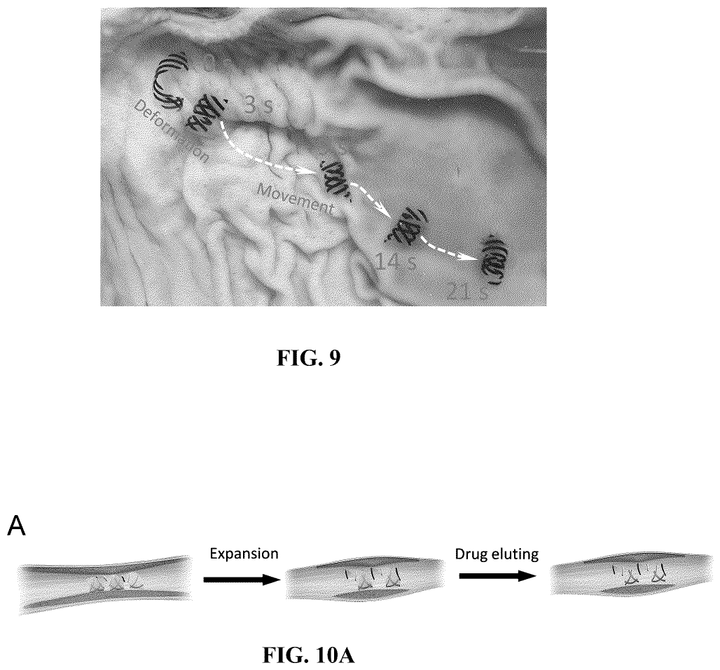

Besides, in order to further explore the potential applications for in vivo biomedical environment of the dual responsive actuator, the real stomach was prepared with simulated stomach fluids. Upon magnetic stimuli, the dual responsive actuator rolled on the wet surface through the viscous fluid, and finally to the wound position within 21 s (FIG. 9). The intriguing reconfiguring and locomotion functions of these actuators offer great potential for applications in harsh environments.

### Example 7—a Soft Drug-Eluting Stent with the Dual Responsive Actuator

Presently, the most effective treatment for coronary artery disease is to implant an expandable vascular stent to widen the narrowed/blocked vessel. For the potential application in biomedical field, a soft drug-eluting stent for artery disease was demonstrated.

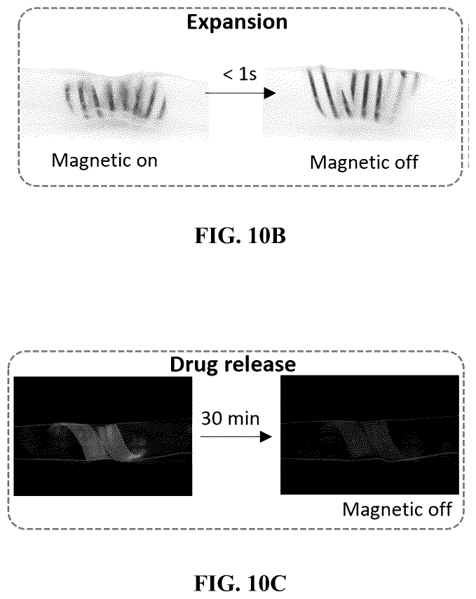

In this example, to validate the designability and feasibility of the dual responsive actuator, a drug eluting vascular stent for coronary artery disease was demonstrated, as shown in FIG. 10A. The reversible deformation of the dual responsive actuator could be used to expand the blood vessel with stenosis under magnetic control. The drug could be loaded on the chitin film and integrate the drug releasing function into the stent.

FIGS. 10A-10B demonstrated the application of dual responsive actuator as a drug eluting vascular stent for coronary artery disease, showing the example to demonstrate the blood vessel expansion and drug releasing functions of the dual responsive actuator. To mimic vascular stenosis, a PDMS tube was created with a decreasing diameter towards to the centre part. The tube was immersed in the 37° C. phosphate-buffered saline (PBS) to mimic the body temperature. After removing the magnetic field, the smaller middle of the tuber quickly expanded to a large diameter within 1 s (FIG. 10B). To test the drug releasing function of the dual responsive actuator, fluorescence dye was loaded into the chitin hydrogel and the change of the fluorescence intensity was recorded (FIG. 10C). The chitin hydrogel started to release the fluorescence dye into the PBS when implanted into the vessel, resulting in a low fluorescence intensity of the hydrogel film after 30 min.

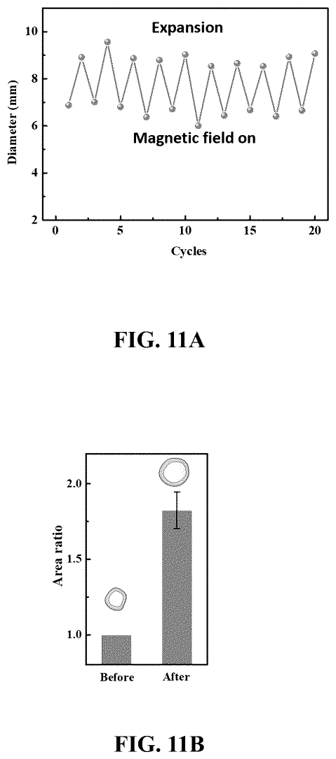

FIGS. 11A-11B showed the deformation characterizations of the dual responsive actuator as a vascular stent. The significant change in the diameter of the vascular from approximately 6 mm to approximately 10 mm before and after expansion were observed, and this rapid and reversible transformation could be repeated on demand by magnetic actuation. The cross-section area of vessel expanded almost 2 times of its original size after implantation of the dual responsive actuator stent, as shown in FIG. 11B.

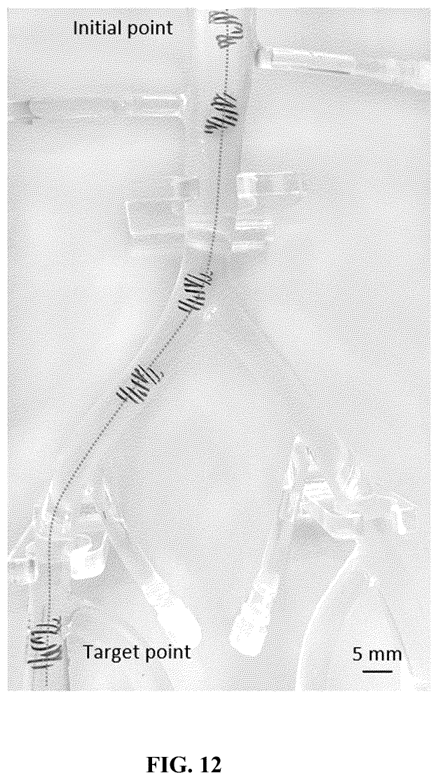

In order to demonstrate the motion control ability of the dual responsive actuator as the vessel stent, a blood vessel model with tortuous structure was used to validate the navigation. Turning to FIG. 12, the dual responsive actuator could move from the initial point to the target point under the control of magnetic field. Thus, with its fast response speed, large expansion ratio, and excellent stability, the dual-responsive actuator emerges as a promising candidate for use as a drug-eluting stent.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to +10% of that numerical value, such as less than or equal to +5%, less than or equal to +4%, less than or equal to +3%, less than or equal to +2%, less than or equal to +1%, less than or equal to +0.5%, less than or equal to +0.1%, or less than or equal to +0.05%.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

It will be appreciated by those skilled in the art, in view of these teachings, that alternative embodiments may be implemented without undue experimentation or deviation from the spirit or scope of the invention, as set forth in the appended claims. This invention is to be limited only by the following claims, which include all such embodiments and modifications when viewed in conjunction with the above specification and accompanying drawings.

### INDUSTRIAL APPLICABILITY

The potential industrial applications of the dual-responsive actuator, particularly as a vascular stent, are currently under scrutiny. The reversible deformation capability of this actuator presents an innovative approach for expanding blood vessels affected by stenosis under the influence of magnetic control. Additionally, the chitin film integrated into the design serves as a versatile platform for drug loading, seamlessly incorporating drug-releasing functionality into the stent. This research holds promise for advancements in medical technology and therapeutic interventions.

### REFERENCES: THE DISCLOSURES OF THE FOLLOWING REFERENCES ARE INCORPORATED BY REFERENCE

1. Chang, C., Chen, S., & Zhang, L. (2011). Novel hydrogels prepared via direct dissolution of chitin at low temperature: structure and biocompatibility. Journal of Materials Chemistry, 21 (11), 3865-3871.

What is claimed is:

1. A 4D-printed humidity and magnetic dual responsive actuator with a bilayer structure, comprising a hydrogel film and a numerous of magnetic elastomer filaments, the numerous of magnetic elastomer filaments are printed on the hydrogel film to formed the bilayer structure, wherein the magnetic elastomer filaments are elastomer filaments embedded with magnetic particles, and the dual responsive actuator capable of responding to both humidity and magnetic fields, resulting in a reversible deformation that transforms into helix structures.

2. The 4D-printed humidity and magnetic dual responsive actuator of claim 1, wherein the hydrogel film comprises chitin, cellulose hydrogel, polyethylene glycol (PEG), poly (N-isopropyl acrylamide) (PNIPAM), polyvinyl alcohol (PVA), methacryloyl-modified gelatin (GelMA), or a combination thereof.

3. The 4D-printed humidity and magnetic dual responsive actuator of claim 1, wherein the magnetic particles comprise neodymium-iron-boron (NdFeB) with a particle size of 5-100 μm.

4. The 4D-printed humidity and magnetic dual responsive actuator of claim 1, wherein the elastomer filaments are polysiloxane materials comprising polydimethylsiloxane, polyborosiloxanes, or polycarbosiloxanes.

5. The 4D-printed humidity and magnetic dual responsive actuator of claim 1, when subjected to external magnetic field control, the dual responsive actuator demonstrates versatile functionality by seamlessly transitioning between two motion modes comprising rolling and wriggling.

6. The 4D-printed humidity and magnetic dual responsive actuator of claim 1, the swelling ratio between the hydrogel film and the magnetic elastomer filaments is 1.07:0.01.

7. The 4D-printed humidity and magnetic dual responsive actuator of claim 1, wherein the dual-responsive actuator is designed for remote operation in a challenging environment comprising enclosed spaces, over high and sloped obstacles, and on viscous stomach surfaces.

8. A method for fabrication a 4D-printed humidity and magnetic dual responsive actuator, comprising the steps of:
preparing a numerous of magnetic elastomer filaments;
printing the numerous of magnetic elastomer filaments on a hydrogel film with predesigned pattern and angles to form printed magnetic elastomer filaments;
curing the printed magnetic elastomer filaments to form a bilayer structure; and
magnetizing the bilayer structure with a predesigned helix structure to obtain the 4D-printed humidity and magnetic dual responsive actuator.

9. The method of claim 8, wherein the hydrogel film comprises chitin, cellulose hydrogel, polyethylene glycol (PEG), poly (N-isopropyl acrylamide) (PNIPAM), polyvinyl alcohol (PVA), methacryloyl-modified gelatin (GelMA), or a combination thereof.

10. The method of claim 8, wherein step of preparing a numerous of magnetic elastomer filaments comprising embedding magnetic particles into elastomer filaments.

11. The method of claim 10, wherein the elastomer filaments are polysiloxane materials comprising polydimethylsiloxane, polyborosiloxanes, or polycarbosiloxanes.

12. The method of claim 10, the magnetic particles comprise neodymium-iron-boron (NdFeB) particles with a particle size of 5 μm to 100 μm.

13. The method of claim 12, the weight ratio of the magnetic particles in the elastomer matrix ranges from 1 wt % to 20 wt %.

14. The method of claim 8, wherein the magnetic elastomer filaments have a printing angle on the hydrogel film ranging from 0° to 90°.

15. The method of claim 8, wherein the printed magnetic elastomer filaments have a diameter of approximately 100 to 500 μm.

16. The method of claim 8, wherein the curing temperature for the bilayer structure ranges from 60° C. to 150° C.

17. The method of claim 8, wherein the swelling ratio between the hydrogel film and the magnetic elastomer filaments is 1.07:0.01.
